(19) United States
(12) Patent Application Publication
(10) Pub. No.: US 2025/0356956 A1
(43) Pub. Date: Nov. 20, 2025

(54) CURRICULUM LEARNING IN FINER SPECTRUM INFERENCE

(71) Applicant: International Business Machines Corporation, Armonk, NY (US)

(72) Inventors: Hajime Shinohara, Yokohama (JP); AKIHIRO KISHIMOTO, Tokyo (JP)

(21) Appl. No.: 18/663,469

(22) Filed: May 14, 2024

(51) Int. Cl. G16C 20/30; G16C 20/70

(52) U.S. Cl. CPC G16C 20/30; G16C 20/70

(57) ABSTRACT

A curriculum learning method yields finer spectrum inference by abstracted an original training dataset. The abstracted training dataset is supplemented with interpolated data points, to create an interpolated abstracted dataset for initial or intermediate machine learning. The final spectrum inference by the training machine learning model is a finer spectrum inference than obtained by individual learning.

### BACKGROUND

The present invention relates generally to the field of chemical and material sciences, and more particularly to accelerated discovery using artificial intelligence (AI) foundation models.

Curriculum learning is a method of training that begins with easier tasks and then trains with more complex tasks. In terms of spectrum analysis, curriculum learning techniques have been used in phonetic sounds analysis. Curriculum learning is also applied to time-series tasks on financial forecasting where the training examples are sorted from the easiest examples to the hardest examples during the training step.

Spectroscopy is a modality for evaluating the properties of materials as the materials interact with or emit electromagnetic radiation. UV spectra, including ultraviolet-visible (UV-vis) spectra, of absorption spectra, enable the measurement of important energy gap structures that provide information about the electronic structure. Spectral features encompass not only direct properties such as peak positions and bandwidths but also quantitative evaluations of features like graph curvature, which can be challenging. This highlights the potential effectiveness of spectroscopy as a modality for machine learning. Absorption spectra show the change in absorbance of a sample as a function of the wavelength of incident light, as measured using a spectrophotometer.

SMILES (Simplified Molecular Input Line Entry System) is a line notation method to represent molecules as well as reactions. SMILES is often used to translate a chemical's three-dimensional structure into a string of symbols that is easily understood by computer software.

Molecular fingerprints are commonly used cheminformatics tools for virtual screening and mapping chemical space. RDKit is an open source toolkit for the cheminformatics field. Among the different types of fingerprints, substructure fingerprints perform best for small molecules such as drugs, while atom-pair fingerprints are preferable for large molecules such as peptides. A popular molecular fingerprint is the Morgan fingerprint, used in small molecule virtual screening and target prediction benchmarks. The Morgan fingerprint perceives the presence of specific circular substructures around each atom in a molecule, which are predictive of the biological activities of small organic molecules. (Note: the term(s) “RDKit” and/or “RDKIT” may be subject to trademark rights in various jurisdictions throughout the world and are used here only in reference to the products or services properly denominated by the marks to the extent that such trademark rights may exist.)

### SUMMARY

In one aspect of the present invention, a method, a computer program product, and a system for inferring spectra data includes: selecting a first spectra dataset from a training dataset, the training dataset having a specified number of data points, the first spectra dataset having fewer data points than the specified number; interpolating the first spectra dataset to generate a first interpolated dataset having the specified number of data points; training an inference model to generate a first inferred spectra dataset from the first interpolated dataset; and causing the trained inference model to generate a final spectrum inference dataset from the training dataset to predict a rough shape of a spectrum.

In a further aspect of the present invention, the method, the computer program product, and the system for inferring spectra data further includes: selecting a second spectra dataset from the training dataset, the second spectra dataset having more data points than the first spectra dataset; interpolating the second spectra dataset to generate a second interpolated dataset having the specified number of data points; and training the inference model to generate a second inferred spectra dataset from the second interpolated dataset. The trained inference model generates a finer spectrum inference dataset to predict a finer shape of the spectrum than the rough shape predicted by the final spectrum inference dataset.

In yet another aspect of the present invention, the method, the computer program product, and the system for inferring spectra data further includes training the trained inference model on the training dataset to leverage the rough shape of the spectrum to enhance accuracy in predicting an overall shape of the spectrum by the trained inference model.

### BRIEF DESCRIPTION OF THE SEVERAL VIEWS OF THE DRAWINGS

FIG. 1 is a schematic view of a first embodiment of a system according to the present invention;

FIG. 2 is a flowchart showing a method performed, at least in part, by the first embodiment system;

FIG. 3 is a schematic view of a machine logic (for example, software) portion of the first embodiment system; and

FIG. 4 is a system flowchart view of a second embodiment of a process according to the present invention.

### DETAILED DESCRIPTION

Spectrum inference, also referred to as spectrum prediction, is a computer-driven technique of inferring the spectrum features from already known, or measured, spectrum statistics by effectively exploiting the inherent correlations among them. Machine learning inference is the process of running data points into a machine learning model to calculate an output such as a single numerical score. The curriculum learning method presented herein provides for machine learning on more easily processed data initially and introducing increasingly more challenging data for processing later. Spectrum data for inference training is often limited in data points. Having limited data points to work with, the curriculum learning process introduced herein advantageously produced interpolated abstracted training datasets drawing from an original training dataset of limited size.

A curriculum learning method yields finer spectrum inference by abstracted an original training dataset. The abstracted training dataset is supplemented with interpolated data points, to create an interpolated abstracted dataset for initial or intermediate machine learning. The final spectrum inference by the training machine learning model is a finer spectrum inference than obtained by individual learning. The present invention may be a system, a method, and/or a computer program product. The computer program product may include a computer readable storage medium (or media) having computer readable program instructions thereon for causing a processor to carry out aspects of the present invention.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not storage in the form of one or more transitory signals, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

Computing environment 100 contains an example of an environment for the execution of at least some of the computer code involved in performing the inventive methods, such as curriculum learning program 300. In addition to block 300, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 300, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

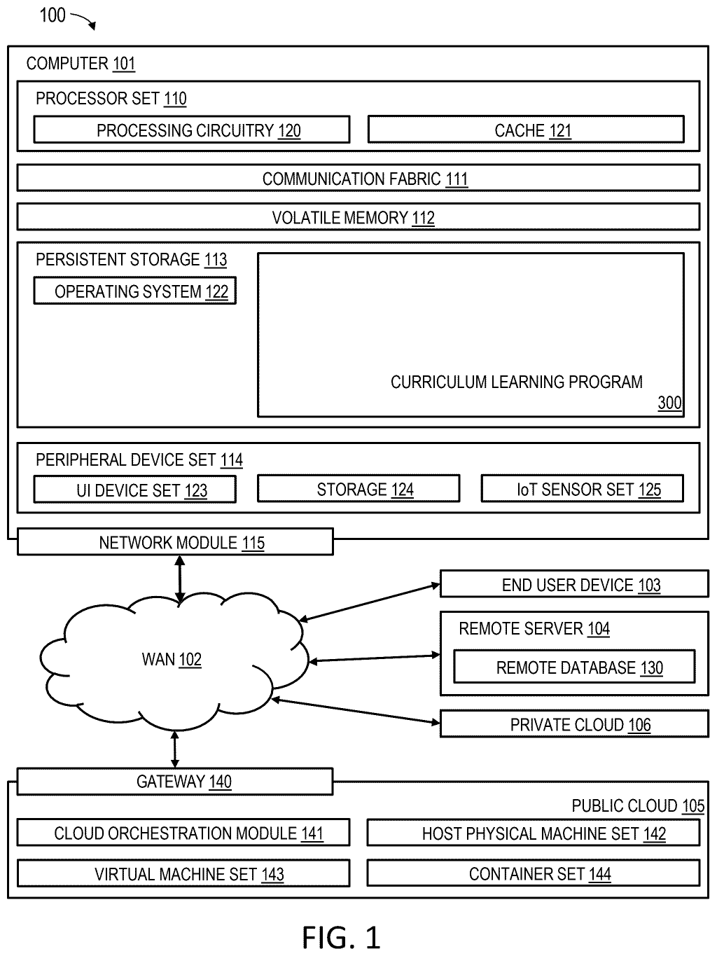

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 300 in persistent storage 113.

COMMUNICATION FABRIC 111 represents the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 300 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The programs described herein are identified based upon the application for which they are implemented in a specific embodiment of the present invention. However, it should be appreciated that any particular program nomenclature herein is used merely for convenience, and thus the present invention should not be limited to use solely in any specific application identified and/or implied by such nomenclature.

Curriculum learning program 300 operates to apply a curriculum learning method to spectrum inference by creating abstract spectra data from an original training dataset. The abstract spectra data is interpolated for initial training of a machine learning model prior to performing training with the original training dataset. Using the abstracted spectra for initial training leverages the rough shape of the spectrum to enhance the accuracy of predicting the overall shape of the spectrum with the original training dataset. Finer training may then be performed using the original spectra data that is not abstracted.

Some embodiments of the present invention recognize the following facts, potential problems and/or potential areas for improvement with respect to the current state of the art: (i) accelerated discovery is relevant for improving artificial intelligence (AI) and/or foundation models for chemical and material sciences; (ii) learning an effective latent representation on molecular structures plays an essential part in many applications such as property prediction and molecular structure generation; (iii) ultraviolet (UV) spectrum is one of the modalities of materials research with which material scientists assess necessary conditions of target materials (e.g., dyes) under development; (iv) due to the experimental difficulties, very little training data is generated; (v) in order to train machine learning models with the small amount of generated data, efficient learning methods are highly sought after; (vi) spectrum prediction is formulated as a sequence prediction problem addressed by approaches such as recurrent neural networks (RNN) and transformer, a deep learning architecture; (vii) models trained without curriculum learning exhibit a problem with generating complicated spectrum as occurs in practice; and/or (viii) the shape of the UV-vis spectra has not been successfully predicted by AI models for many substances.

Some embodiments of the present invention are directed to training a machine learning model to infer a spectrum using curriculum learning methods such that the accuracy of spectrum prediction is improved. Some embodiments of the present invention are directed to curriculum learning methods for a UV spectrum where the overall spectrum feature effects specific peak positions. Focusing on the overall spectrum feature, abstracting the spectrum, and roughly training the model first is an effective training technique for finer spectrum inference.

Because spectrum information is related to various material properties, such as the HOMO-LUNO (highest occupied molecular orbital-least unoccupied molecular orbital) energy gap and color of the materials, scientists and informaticians can use the UV spectrum generated by embodiments of the present invention to evaluate or estimate the properties of various materials.

Some embodiments of the present invention are directed to a process including: (i) applying a curriculum learning method to training a model in finer spectrum inference; (ii) automatically creating abstract spectra with interpolations, given a training dataset; and (iii) using abstracted spectra for initial training, then using the original spectra, which are not abstracted, for finer training. This step effectively leverages the rough shape of the spectra to enhance the accuracy of predicting the overall shape of the spectrum.

Some embodiments of the present invention are directed to a curriculum training method for machine learning models, which involves training on easier tasks first and, then, further training on increasingly complex tasks.

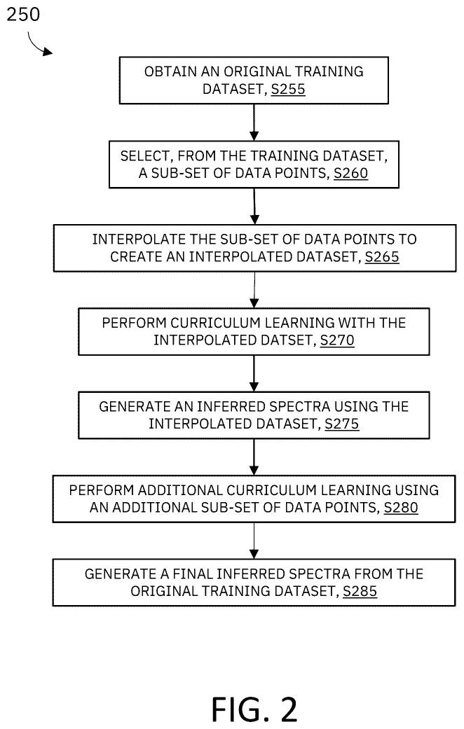

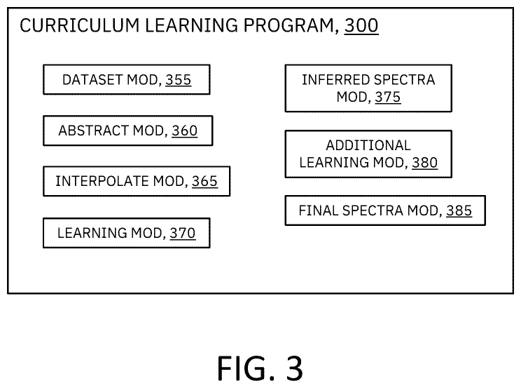

FIG. 2 shows flowchart 250 depicting a first method according to the present invention. FIG. 3 shows program 300 for performing at least some of the method steps of flowchart 250. This method and associated software will now be discussed, over the course of the following paragraphs, with extensive reference to FIG. 2 (for the method step blocks) and FIG. 3 (for the software blocks).

Processing begins at step S255, where dataset module (“mod”) 355 obtains an original training dataset. In this example, the training dataset includes molecule information in the form of a set of SMILES (Simplified Molecular Input Linc Entry System) strings and spectrum data points, the data points being labelled as the SMILES strings. The training datasets may be obtained from research projects within a knowledge domain of interest. An original training dataset may be obtained from an RDKit including SMILES expressions, including morgan fingerprints. For example, evaluation of the performance improvements available upon implementation of aspects of the present invention is performed using an existing research dataset referred to as a UV-adVISor dataset. (Note: the term(s) “UV-adVisor” and/or “UV-ADVISOR” may be subject to trademark rights in various jurisdictions throughout the world and are used here only in reference to the products or services properly denominated by the marks to the extent that such trademark rights may exist.)

Processing proceeds to step S260, where abstract mod 360 selects, from the training dataset, a sub-set of data points. The sub-set of data points is also referred to herein as abstracted spectra data. The selection of data points varies according to the particular training model. For example, a training dataset of 171 data points may be abstracted as three different datasets containing different percentages of original data points. The dataset having the fewest original data points, the remaining being interpolated, is the easier dataset for learning.

Processing proceeds to step S265, where interpolate mod 365 interpolates the sub-set of data points to create an interpolated abstracted dataset. In this example, the interpolated data points are added to the selected data points such that the combined total data points is equal to the number of data points in the original training dataset. Alternatively, the combined total data points varies from the number of data points in the original training dataset. The total number of data points may be determined by policy and the percentage of total data points that are interpolated may also be pre-defined according to policy. It should be noted that the curriculum learning process generally uses easier to infer datasets for initial training. For example, the dataset having fewer original training set data points. Datasets generated from abstracted training data that includes interpolated data points is generally easier to generate an inferred spectra than the original training datasets.

Processing proceeds to step S270, where learning mod 370 performs curriculum learning on a machine learning model with the interpolated abstracted dataset. During learning, the machine learning model outputs inferred spectra based on the interpolated abstracted dataset. The likelihood of making a correct inference depends on the number of interpolated data points included in the interpolated abstracted dataset. Additional rounds of curriculum learning are performed in subsequent steps to yield finer spectrum inference. The additional rounds, or learning loops, are performed on increasingly difficult-to-infer datasets according to the curriculum learning plan.

Processing proceeds to step S275, where inferred spectra mod 375 generated an inferred spectra using the interpolated abstracted dataset. In this example, the inferred spectra are the original size spectra because the interpolated abstracted dataset is created to have the same number of data points as the original training dataset.

Processing proceeds to step S280, where additional learning mod 380 performs additional curriculum learning on the machine learning model using an additional sub-set of data points. In this example, the additional sub-set of data points is selected from the original training dataset and includes a higher percentage of original data points than the previously selected sub-set of data points. Alternatively, the additional sub-set of data points is derived from the inferred spectra generated at step S275.

Processing ends at step S285, where final spectra mod 385 generates a final inferred spectra from the original training dataset. In this example, the original training dataset from which the final inferred spectra is generated does not include any interpolated data. Further, in this example, the original training dataset is not abstracted when generating the final inferred spectra. As discussed herein, the final inferred spectra, when generated by a machine learning model trained according to a curriculum learning process as disclosed herein, yields a finer spectrum inference than when generated by a conventionally trained model using individual learning.

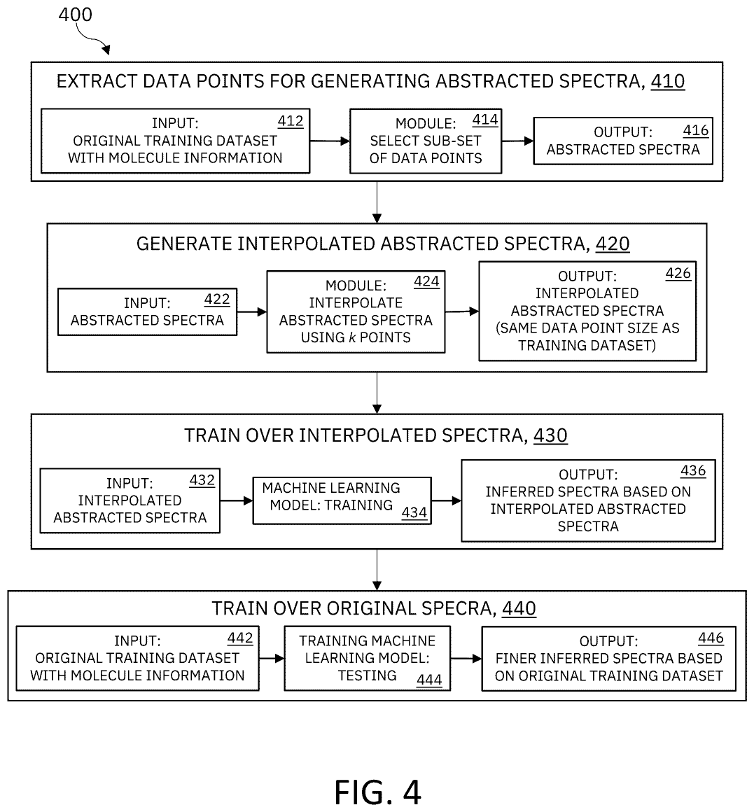

Further embodiments of the present invention are discussed in the paragraphs that follow, starting with FIG. 4, illustrating a process according to some embodiments of the present invention.

FIG. 4 is a system flow diagram of process 400 including 4 sub-processes, 410, 420, 430, and 440. Process 400 may be performed by curriculum learning program 300 in computing environment 100 (FIG. 1).

Processing begins at step 410, where data points are extracted for generating abstracted spectra. The process of extracting the data points includes: (i) input 412, the original training dataset with molecule information; (ii) module 414 to select a sub-set of data points, k, from the original spectra data; and (iii) output 416, the abstracted spectra data making up the selected sub-set, k, from the original spectra data. Some embodiments of the present invention abstract various datasets from the original dataset. For example, one abstracted dataset may include 75 percent of the original data points and another abstracted dataset may include only half of the original data points. The balance of the data points being interpolated, as described below.

Processing proceeds to step 420, where interpolated abstracted spectra data are generated. The process of generating the interpolated abstracted spectra data includes: (i) input 422, the abstracted spectra data from step 410; (ii) module 424 to interpolate the abstracted spectra using k points; and (iii) output 426, the interpolated abstracted spectra having the same data point count as the original training dataset.

Processing proceeds to step 430, where the machine learning model is trained over the interpolated abstracted spectra data. The process of training the machine learning model includes: (i) input 432, the interpolated abstracted spectra data from step 420; (ii) training the machine learning model, 434; and (iii) output 436, the inferred spectra based on the interpolated abstracted spectra data, which is trained over the interpolated abstracted spectra data with the original size spectra training dataset.

Processing proceeds to step 440, where the machine learning model is trained over the original spectra training dataset. The process of training over the original dataset includes: (i) input 442, the original training dataset having, for example, spectrum data points labelled as SMILES strings; (ii) trained model testing 444, with original spectra data; and (iii) output 446, the finer inferred spectra data based on the original training dataset. Where the inferred spectra data is the same size as the original spectra.

According to some embodiments of the present invention, the following tools/techniques may be deployed, including: (a) using RDKit for handling simplified molecular input line entry system (SMILES) expressions (e.g. morgan fingerprints); (b) using a numpy interpolation module for spectra data point interpolation; (c) using a fully connected neural network unit (FCU) model; and (d) using a root mean square error (RMSE) loss function. The numpy interpolation module, numpy.interp, is a one-dimensional linear interpolation for monotonically increasing sample points. It returns the one-dimensional piecewise linear interpolant to a function.

When a spectrum inference model is trained according to a curriculum learning method defined by some embodiments of the present invention, the trained inference model can be used to infer UV spectrum in chemical science and/or material science. For example, given a training dataset consisting of SMILES expressions, the trained spectrum inference model can generate a model for predicting finer UV spectrum parameters, including overall shape of the spectrum, as follows: (i) prepare abstracted spectra data points from given UV spectra data; (ii) for each training example, SMILES fingerprints and abstracted spectra data are used as input and output, respectively; and (iii) for each training loop, the inference model is trained with the latest abstracted spectra data where the balance of data points between the abstracted spectra points due to the abstraction process are added by linear interpolation of the abstracted data points. In that way, the inference model continues to be trained by the same number of data points as the original spectra data without abstraction.

According to some embodiments of the present invention, predicting a finer UV absorption spectrum may be performed. For example, given a solute and a solvent, predict finer UV absorption spectrum. This task of finer UC spectra inference plays an important role in material discovery. In order to evaluate the use of curriculum learning against conventional individual learning methods, a spectra dataset is needed. The spectra datasets used in the evaluation described below originated from research in the spectrum inference domain and is referred to as the UV-adVISor dataset, where a set of SMILES string and spectrum data points are created. The spectra dataset includes 171 data points of 230-400 nm. A total of 3,170 molecules were prepared to perform the finer UV spectra inference task. The spectra datasets used for the evaluation task are set up according to some embodiments of the present invention in that the spectrum data points included in the datasets are labelled as the SMILES string. Of the 3,170 molecules, 2,536 molecules were used for training, 317 molecules were used for validation, and 317 molecules were used for testing.

Four spectra datasets were prepared from the UV-adVISor dataset. The four training datasets of 171 data points were set up as follows: 1) dataset 22, in which 1 in 8 original data points are interpolated to make 171 total data points; (2) dataset 43, in which 1 in 4 original data points are interpolated; and (3) dataset 86, in which 1 in 2 original data points are interpolated; and, finally, (4) the original dataset, referred to as dataset 171, with all 171 data points being original training data points.

The evaluation process was performed as follows: (i) the spectrum inference models were trained by a training dataset, their hyperparameters were optimized with a validation dataset, and their performances were tested by a test dataset; (ii) the final loss was obtained after the final 1000^{th} epoch; and (iii) the training loss and validation loss were smoothed with window size 20 to be able to reduce the noise of the loss. The RMSE values are listed in Table 1 as Test Loss for each corresponding validation set obtained by neural network models (e.g. FCU) receiving inferred UV spectra data. The summary of each model's different curriculum learning data points are illustrated in Table 1, below. The numbers of the models show the data points of the spectrum.

TABLE 1
Test Loss Comparison for Curriculum Trained Models.

| | Test |
|---|---|
| Learning Model | Loss |
| 171 (individual) | 0.7968 |
| 22-171 (Single-Loop Curriculum) | 0.7777 |
| 43-171 (Single-Loop Curriculum) | 0.7913 |
| 86-171 (Single-Loop Curriculum) | 0.7956 |
| 22-43-171 (Double-Loop Learning Curriculum) | 0.7934 |
| 43-86-171 (Double-Loop Learning Curriculum) | 0.7782 |
| 22-43-86-171 (Triple-Loop Learning Curriculum) | 0.7918 |

Each of the curriculum learning models have lower test losses than the individual learning model 171, using only dataset 171. This indicates that the additional steps in the curriculum improve spectrum inference performance. Multi-step, or multi-loop, curriculum learning such as learning model 43-86-171, a triple-loop learning model, also has lower test losses than the individual learning model 171, which indicates the additional steps in the curriculum improve performance. The curriculum learning model 22-43-86-171 trains first using a dataset having 22 original data points that are interpolated (for easier inference), then model 43 is used, with 43 original data points being interpolated. The model 22-43-86-171 then uses model 86 for training, with 86 original data points, the second most original data points, and, finally, learning is performed using model 171, with all 171 data points being original data points. With individual learning model 171 presenting a 0.7968 test loss, all of the other learning models exhibited less test loss. Specifically, model 22-171 and model 43, 86, 171 exhibited the best performance, with only 0.778 test loss each. The test loss values show that curriculum learning has advantages in terms of model performance when compared to the individual learning model. As demonstrated by the finer UV spectra inference task, a cumulative learning model using increasingly difficult datasets improves the performance of spectrum inference by machine learning models.

Some embodiments of the present invention may include one, or more, of the following features, characteristics and/or advantages: (i) does not need solid state chemistry information such as atomic coordination and point groups; (ii) focus on UV spectrum and organic chemical materials and do not need some solid-state chemistry information such as atomic coordination and point groups; (iii) involves domain specific knowledge of spectrum specific features (such as curvature of spectrum in materials science and stellar atmospheres in chemical science) for predicting spectrum by their model; (iv) includes the algorithmic component of abstraction to be able to perform curriculum learning; (v) an intermediate model that can infer more precise spectrum features; (vi) an intermediate model that can efficiently infer spectrum features with a limited amount of data; (vii) chemical property prediction; (viii) molecular structure generation; and/or (iv) molecular structure optimization.

Some embodiments of the present invention are directed to training a spectrum inference model used for generating a spectrum that includes: (i) a training dataset containing molecule description (e.g., SMILES) and spectra data; (ii) extracting a subset of data points from spectra data to generate abstracted spectrum; (iii) interpolating additional data points in the abstracted spectra data to generate an interpolated abstracted spectra dataset; and/or (iv) training the spectrum inference model according to a loss function with the interpolated abstracted spectra data as well as original spectra data in the training dataset.

Some embodiments of the present invention are directed to training a machine learning model for generating a UV spectrum and generating abstracted spectra from the UV spectrum.

Some embodiments of the present invention are directed to training a machine learning model according to a loss function accounting for a comparison between the input and the output of the model (e.g., RMSE loss).

Some embodiments of the present invention are directed to training a spectrum inference model consisting of a neural network (e.g., Fully Connected Unit (FCU)).

Some embodiments of the present invention are directed to interpolating data points for an abstracted spectra dataset to generate an interpolated abstracted spectrum, where interpolating the data points is performed with linear interpolation.

Some helpful definitions follow:

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein that are believed as maybe being new, this understanding, as indicated by use of the term “present invention,” is tentative and provisional and subject to change over the course of patent prosecution as relevant information is developed and as the claims are potentially amended.

Embodiment: see definition of “present invention” above-similar cautions apply to the term “embodiment.”

and/or: inclusive or; for example, A, B “and/or” C means that at least one of A or B or C is true and applicable.

User/subscriber: includes, but is not necessarily limited to, the following: (i) a single individual human; (ii) an artificial intelligence entity with sufficient intelligence to act as a user or subscriber; and/or (iii) a group of related users or subscribers.

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

In a nutshell, the inventive concept can be summarized by the following clauses:

1. A computer-implemented method for inferring spectra data, the method comprising:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset; and
causing the trained inference model to generate a final spectrum inference dataset from the training dataset to predict a rough shape of a spectrum.

2. The computer-implemented method of clause 1, further comprising:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

3. The computer-implemented method of clause 2, wherein the first spectra dataset includes fewer than half the specified number of data points; and the second spectra dataset includes more than half the specified number of data points.

4. The computer-implemented method of claim 1, further including obtaining the training dataset including spectrum data points labelled as simplified molecular input line entry system (SMILES) strings.

5. The computer-implemented method of clause 1, wherein the training is performed according to a loss function accounting for a comparison between the first spectra dataset and the spectrum inference dataset.

6. The computer-implemented method of clause 1, further comprising training the trained inference model on the training dataset to leverage the rough shape of the spectrum to enhance accuracy in predicting an overall shape of the spectrum by the trained inference model.

7. The computer-implemented method of clause 1, wherein the interpolating is performed with linear interpolation.

8. The computer program product comprising a computer-readable storage medium having a set of instructions stored therein which, when executed by a processor, causes the processor to infer spectra data by:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset; and
causing the trained inference model to generate a final spectrum inference dataset from the training dataset to predict a rough shape of a spectrum.

9. The computer program product of clause 8, the set of instructions further causing the processor to infer spectra data by:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

10. The computer program product of clause 8, wherein the first spectra dataset includes fewer than half the specified number of data points; and the second spectra dataset includes more than half the specified number of data points.

11. The computer program product of clause 8, the set of instructions further causing the processor to infer spectra data by obtaining the training dataset including spectrum data points labelled as simplified molecular input line entry system (SMILES) strings.

12. The computer program product of clause 8, wherein the training is performed according to a loss function accounting for a comparison between the first spectra dataset and the spectrum inference dataset.

13. The computer program product of clause 8, the set of instructions further causing the processor to infer spectra data by training the trained inference model on the training dataset to leverage the rough shape of the spectrum to enhance accuracy in predicting an overall shape of the spectrum by the trained inference model.

14. A computer system for inferring spectra data, the computer system comprising:
a processor set; and a computer readable storage medium; wherein the processor set is structured, located, connected, and/or programmed to run program instructions stored on the computer readable storage medium; and the program instructions which, when executed by the processor set, cause the processor set to infer spectra data by:
   selecting a first spectra dataset from a training dataset, the training dataset having a specified number of data points, the first spectra dataset having fewer data points than the specified number;
   interpolating the first spectra dataset to generate a first interpolated dataset having the specified number of data points;
   training an inference model to generate a first inferred spectra dataset from the first interpolated dataset; and
   causing the trained inference model to generate a final spectrum inference dataset from the training dataset to predict a rough shape of a spectrum.

15. The computer system of clause 14, further causing the processor set to infer spectra data by:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

16. The computer system of clause 14, wherein the first spectra dataset includes fewer than half the specified number of data points; and the second spectra dataset includes more than half the specified number of data points.

17. The computer system of clause 14, further causing the processor set to infer spectra data by obtaining the training dataset including spectrum data points labelled as simplified molecular input line entry system (SMILES) strings.

18. The computer system of clause 14, wherein the training is performed according to a loss function accounting for a comparison between the first spectra dataset and the spectrum inference dataset.

19. The computer system of clause 14, further causing the processor set to infer spectra data by training the trained inference model on the training dataset to leverage the rough shape of the spectrum to enhance accuracy in predicting an overall shape of the spectrum by the trained inference model.

20. The computer system of clause 14, wherein the interpolating is performed with linear interpolation.

21. A computer-implemented method for inferring spectra data, the method comprising:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset;
causing the trained inference model to generate a final spectrum inference dataset from the training dataset to predict a rough shape of a spectrum.
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

22. A computer-implemented method for inferring spectra data, the method comprising:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset;
causing the trained inference model to generate a final spectrum inference dataset from the training dataset to predict a rough shape of a spectrum; and
training the trained inference model on the training dataset to leverage the rough shape of the spectrum to enhance accuracy in predicting an overall shape of the spectrum by the trained inference model.

23. A computer-implemented method for inferring spectra data, the method comprising:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset;
causing the trained inference model to generate a final spectrum inference dataset from the training dataset to predict a rough shape of a spectrum.
selecting a second spectra dataset from the training dataset, the second spectra dataset having more data points than the first spectra dataset;
interpolating the second spectra dataset to generate a second interpolated dataset having the specified number of data points;
training the inference model to generate a second inferred spectra dataset from the second interpolated dataset, wherein the trained inference model generates a finer spectrum inference dataset to predict a finer shape of the spectrum than the rough shape predicted by the final spectrum inference dataset; and
training the trained inference model on the training dataset to leverage the rough shape of the spectrum to enhance accuracy in predicting an overall shape of the spectrum by the trained inference model.

What is claimed is:

1. A computer-implemented method for inferring spectra data, the method comprising:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset; and
causing the trained inference model to generate a final spectrum inference dataset from the training dataset to predict a rough shape of a spectrum.

2. The computer-implemented method of claim 1, further comprising:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

3. The computer-implemented method of claim 2, wherein:
the first spectra dataset includes fewer than half the specified number of data points; and
the second spectra dataset includes more than half the specified number of data points.

4. The computer-implemented method of claim 1, further including:
obtaining the training dataset including spectrum data points labelled as simplified molecular input line entry system (SMILES) strings.

5. The computer-implemented method of claim 1, wherein the training is performed according to a loss function accounting for a comparison between the first spectra dataset and the spectrum inference dataset.

6. The computer-implemented method of claim 1, further comprising:
training the trained inference model on the training dataset to leverage the rough shape of the spectrum to enhance accuracy in predicting an overall shape of the spectrum by the trained inference model.

7. The computer-implemented method of claim 1, wherein the interpolating is performed with linear interpolation.

8. A computer program product comprising a computer-readable storage medium having a set of instructions stored therein which, when executed by a processor, causes the processor to infer spectra data by:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset; and
causing the trained inference model to generate a final spectrum inference dataset from the training dataset to predict a rough shape of a spectrum.

9. The computer program product of claim 8, the set of instructions further causing the processor to infer spectra data by:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

10. The computer program product of claim 8, wherein:
the first spectra dataset includes fewer than half the specified number of data points; and
the second spectra dataset includes more than half the specified number of data points.

11. The computer program product of claim 8, the set of instructions further causing the processor to infer spectra data by:
obtaining the training dataset including spectrum data points labelled as simplified molecular input line entry system (SMILES) strings.

12. The computer program product of claim 8, wherein the training is performed according to a loss function accounting for a comparison between the first spectra dataset and the spectrum inference dataset.

13. The computer program product of claim 8, the set of instructions further causing the processor to infer spectra data by:
training the trained inference model on the training dataset to leverage the rough shape of the spectrum to enhance accuracy in predicting an overall shape of the spectrum by the trained inference model.

14. A computer system for inferring spectra data, the computer system comprising:
a processor set; and
a computer readable storage medium;
wherein:
the processor set is structured, located, connected, and/or programmed to run program instructions stored on the computer readable storage medium; and
the program instructions which, when executed by the processor set, cause the processor set to infer spectra data by:
selecting a first spectra dataset from a training dataset, the training dataset having a specified number of data points, the first spectra dataset having fewer data points than the specified number;
interpolating the first spectra dataset to generate a first interpolated dataset having the specified number of data points;
training an inference model to generate a first inferred spectra dataset from the first interpolated dataset; and
causing the trained inference model to generate a final spectrum inference dataset from the training dataset to predict a rough shape of a spectrum.

15. The computer system of claim 14, further causing the processor set to infer spectra data by:
selecting a second spectra dataset from the training dataset, the second spectra dataset having more data points than the first spectra dataset;
interpolating the second spectra dataset to generate a second interpolated dataset having the specified number of data points; and
training the inference model to generate a second inferred spectra dataset from the second interpolated dataset;
wherein the trained inference model generates a finer spectrum inference dataset to predict a finer shape of the spectrum than the rough shape predicted by the final spectrum inference dataset.

16. The computer system of claim 14, wherein:
the first spectra dataset includes fewer than half the specified number of data points; and
the second spectra dataset includes more than half the specified number of data points.

17. The computer system of claim 14, further causing the processor set to infer spectra data by:
obtaining the training dataset including spectrum data points labelled as simplified molecular input line entry system (SMILES) strings.

18. The computer system of claim 14, wherein the training is performed according to a loss function accounting for a comparison between the first spectra dataset and the spectrum inference dataset.

19. The computer system of claim 14, further causing the processor set to infer spectra data by:
training the trained inference model on the training dataset to leverage the rough shape of the spectrum to enhance accuracy in predicting an overall shape of the spectrum by the trained inference model.

20. The computer system of claim 14, wherein the interpolating is performed with linear interpolation.
